(19) United States
(12) Patent Application Publication
(10) Pub. No.: US 2012/0101511 A1
(43) Pub. Date: Apr. 26, 2012

(54) Medical Apparatus for Dissociation of Epidural Adhesion and Diminution of Stenosis of Intervertebral Foramen

(76) Inventors: Myungji You, Seoul (KR); Hyunhong Kim, Seoul (KR)

(21) Appl. No.: 13/277,346

(22) Filed: Oct. 20, 2011

(30) Foreign Application Priority Data
Oct. 21, 2010 (KR) 10-2010-0103146

(51) Int. Cl. A61B 17/22

(52) U.S. Cl. 606/159

(57) ABSTRACT

Provided is a medical apparatus which may be used for a patient with low back pain according to epidural adhesion and stenosis of the intervertebral foramen. The medical apparatus includes an insertion tube in which a balloon part, a movable end, and the like are selectively installed in order to alleviate epidural adhesion and stenosis of the intervertebral foramen, which are responsible for low back pain.

### CROSS-REFERENCE TO RELATED APPLICATIONS

This application is based on and claims priority from Korean Patent Application No. 10-2010-0103146, filed on Oct. 21, 2010, with the Korean Intellectual Property Office, the disclosure of which is incorporated herein in its entirety by reference.

### TECHNICAL FIELD

The present disclosure relates to a medical apparatus for dissociation of epidural adhesion and diminution (hereinafter, referred to as “alleviation”) of stenosis of the intervertebral foramen, and more specifically, to a medical apparatus including an insertion tube in which a balloon part, a movable end, and the like are selectively installed.

The present disclosure relates to an external fluid supply unit which supplies fluid to a balloon part.

### BACKGROUND

The “low back pain” generally refers to pain felt in the waist and legs. Low back pain is one of the most common disorders that can be experienced by an individual during his or her lifetime, and a common symptom second to a cold, which causes adults at the age of 55 or less to miss work. Low back pain ranks the second to headaches as the most common reason for chronic pain.

Recently, as the rates of spinal surgery drastically increase, the number of patients with so-called failed back surgery syndrome, who has recurring or aggravated low back pain after spinal surgery, also increases. The adhesions and fibrosis at a site outside the dura surrounding the spinal cord, that is, the epidural space, are known to be mainly responsible for pain from the syndrome. Epidural space refers to the space between the dura which is a protective membrane surrounding the spinal cord and the spinal canal, and is a tissue that is replete with nerve fibers, adipose tissues, connective tissues, blood vessels, and lymphatic vessels. Detailed arrangements thereof are shown in FIGS. 9, 10A, and 10B. As shown in FIG. 10B, the epidural space can be divided into the anterior epidural space and the posterior epidural space. The anterior epidural space is surrounded by the yellow ligament and the periosteum of the vertebral body, the centrum, the intervertebral disc, and the posterior longitudinal ligament are positioned on the front side thereof, the dura is positioned on the rear side thereof, and the anteroposterior diameter is 1.3 to 2.2 mm at the lumbar region. The anteroposterior diameter of the posterior epidural space is known to be about 2.0 to 6.0 mm

In many cases, epidural adhesion occurs after spinal surgery. However, the epidural adhesion often occurs in herniated intervertebral discs, degenerative spinal stenosis, and the like. In the herniated intervertebral discs, nucleus pulpous in discs escapes at either sides or rear through a tear in the posterior annulus fibrosis, which cause cellular deposition and inflammatory response in the epidural space, thereby generating adhesion, while spinal stenosis occurs, such that adhesion is caused by inflammatory mediators. It is thought that patients with chronic low back pain, regardless of its causes, are suffering from adhesions in most cases. The presence of adhesions in the epidural space may cause inflammation of the nerve root in the epidural space and interfere with the mobility of the dural sleeve to cause pain. These adhesions interfere with venous blood flow of the epidural space resulting in edema of the nerve root. Thus, adhesions between the dura and the posterior longitudinal ligament may be responsible for pain and chemical stimuli of the dura and the nerve root may be responsible for the pain. That is, inflammatory inducers, such as substance P, calcium gene-related peptide, c-fos, phospholipase A2, are present in abnormal amounts in the anterior epidural space of a compressed neural tissue, thereby causing inflammatory responses and pain.

Another major contributor to low back pain is stenosis of the intervertebral foramen caused by narrowing of the intervertebral foramen through which the vertebral canal or nerves pass by the facet joints, the yellow ligament, the discs, and the like, which are constituting the spinal canal.

When low back pain is caused by these two disorders, severe pain is felt in the waist and lower limbs. In some cases, an individual cannot walk far or for a long time.

The best treatment for these two disorders is relief of compression on the nerves and blood vessels by removing adhesions in epidural adhesions and widening stenotic sites in stenosis of the intervertebral foramen. Of course, if the treatment is performed through surgery, it is advantageous in that the fundamental cause of the symptom may be removed. However, the treatment costs a lot, and surgery failures and the recurrence of the disease are high. Thus, non-surgical methods have recently been tried.

As shown in FIG. 11, there is a representative method for treating the symptom by inserting a needle into an affected epidural space or transforaminal space of the spine to inject a drug that inhibits inflammation or edema. However, when a drug is not delivered or spread in a sufficient amount to a target in a patient with adhesions and when fibrosis has progressed due to severe adhesions, there are limitations in alleviating the symptom due to little response to anti-inflammatory actions of steroids, and thus a treatment result is not sufficient. Therefore, there is need for a medical apparatus for alleviating epidural adhesion and stenosis of the intervertebral foramen more effectively and safely in a clinical setting.

### SUMMARY

The present disclosure has been made in an effort to provide a medical apparatus for dissociation of epidural adhesion and alleviation of stenosis of the intervertebral foramen, which are both responsible for low back pain, as a medical apparatus which can be used for a patient with low back pain due to epidural adhesion and stenosis of the intervertebral foramen.

An exemplary embodiment of the present disclosure provides a medical apparatus for dissociation of epidural adhesion and alleviation of stenosis of the intervertebral foramen, the apparatus includes: an insertion tube configured to include a first end to be inserted in vivo and a second end that is opposed to the first end, and in which a through hole extending from the first end to the second end is formed therein; a balloon part formed on an outer side of the insertion tube, being spaced apart at a predetermined distance from the first end of the insertion tube and configured to be inflatable as a fluid is supplied; and a fluid injection tube installed within a wall of the insertion tube along a longitudinal direction of the insertion tube and configured to supply the fluid to the balloon part by communicating the balloon part with a fluid supply source.

The medical apparatus of the present disclosure may further include: a pair of wires attached around the first end of the insertion tube and extending toward the second end of the insertion tube. A portion of the insertion tube from the first end of the insertion tube to the balloon part may form a movable end, such that the movable end is bent to one side when one of the pair of wires is pulled, and the movable end is bent to the other side when the other of the pair of wires is pulled.

In the medical apparatus of the present disclosure, the pair of wires may extend through the inside of the wall of the insertion tube.

In the medical apparatus of the present disclosure, the movable end may be contemplated to return to an original position by a restoring force of a spring which is installed at an internal portion of the balloon part if none of the wires is pulled. The spring may be formed of a coil spring, and a through hole of the insertion tube may pass through the inside of the coil spring.

The medical apparatus of the present disclosure may further include: a main body that is connected to the insertion tube at the second end of the insertion tube; and a dial which is rotatably connected to the main body and to which a pair of wires are connected. The pair of wires may be attached to the dial such that any one of the pair of wires is selectively pulled according to rotation in one direction of the dial.

The medical apparatus of the present disclosure may further include a fixing switch which is installed on the main body and can fix a rotation of the dial.

In the medical apparatus of the present disclosure, the through hole of the insertion tube may be for injecting a drug or inserting a catheter. For this purpose, a hole for spraying a drug, communicating with the through hole, may be formed at the first end of the insertion tube and a side wall around the first end of the insertion tube.

The medical apparatus of the present disclosure may further include: a main body connected to the insertion tube at the second end; a conduit provided to the main body and is connected to the second end of the insertion tube, and the conduit may be used for injecting a drug into a through hole of the insertion tube or inserting a catheter.

Another exemplary embodiment of the present disclosure provides a medical apparatus for dissociation of epidural adhesion and alleviation of stenosis of the intervertebral foramen, the apparatus including: a catheter configured to include a first end to be inserted in vivo and a second end that is opposite to the first end, and in which a through hole extending from the first end to the second end is formed therein; a balloon part formed on an outer side of the catheter, being spaced apart at a predetermined distance from the first end of the catheter and configured to be inflatable as a fluid is supplied; and a fluid injection tube installed in a wall of the catheter along a longitudinal direction of the catheter and configured to supply the fluid to the balloon part by communicating the balloon part with a fluid supply source.

The fluid supply source which is allowed to communicate with the fluid injection tube of the medical apparatus may be an external fluid supply unit that includes an injector, a piston slidably movable within the injector, and a connecting part that is detachable to the fluid injection tube. The piston of the external fluid supply unit may be controlled to preset an internal volume of the external fluid supply unit before the connecting part of the external fluid supply unit is connected to the fluid injection tube, the external fluid supply unit and the balloon part are connected to each other through the fluid injection tube, and a volume of the fluid is supplied to the balloon part by an amount decreased from the internal volume of the external fluid supply unit to inflate the balloon part. The external fluid supply unit may further include a blocking valve capable of blocking communication with the fluid injection tube. The external fluid supply unit may further include a spring which pushes a piston in a direction of increasing an internal volume of the injector.

The medical apparatus of the present disclosure may have a hole for spraying a drug, which is communicated with the through hole, formed at a first end of the catheter and a side wall around the first end of the catheter. A hole for injecting a drug may be connected to a second end of the catheter such that the hole may communicate with the through hole of the catheter. A fluid injection tube may extend from the second end of the catheter to the outside of the catheter and a connecting part connected to an external fluid supply unit may be formed at an extended end of the fluid injection tube.

According to exemplary embodiments of the present disclosure, in order to alleviate epidural adhesion and stenosis of the intervertebral foramen, it is possible to alleviate the adhesion and stenosis physically with a balloon part, a movable end, and the like, while allowing a catheter to be inserted in vivo and left therein such that a therapeutic agent may be administered for several days, thereby maximizing the therapeutic effect on low back pain.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a top view of a medical apparatus according to a first exemplary embodiment of the present disclosure.

FIG. 2 is a side view of a medical apparatus according to the first exemplary embodiment of the present disclosure.

FIGS. 3A to 3C are magnifying views of an insertion tube end of a medical apparatus according to the first exemplary embodiment of the present disclosure.

FIGS. 4A and 4B are magnifying views showing an operation of an insertion tube end of a medical apparatus according to the first exemplary embodiment of the present disclosure.

FIG. 5 is a view showing the use state of a medical apparatus according to the first exemplary embodiment of the present disclosure.

FIG. 6 is a perspective view of an insertion tube guide used to assist a medical apparatus according to the first exemplary embodiment of the present disclosure.

FIG. 7 is a view showing the use state of the insertion tube guide used to assist a medical apparatus according to the first exemplary embodiment of the present disclosure.

FIG. 8 is a view showing a medical apparatus according to a second exemplary embodiment of the present disclosure.

FIG. 9 is a view showing a position relationship between dura and epidural space.

FIGS. 10A and 10B are views showing position relationships of epidural space.

FIG. 11 is a view showing insertion positions of the medical apparatus.

### DETAILED DESCRIPTION

In the following detailed description, reference is made to the accompanying drawing, which form a part hereof. The illustrative embodiments described in the detailed description, drawing, and claims are not meant to be limiting. Other embodiments may be utilized, and other changes may be made, without departing from the spirit or scope of the subject matter presented here.

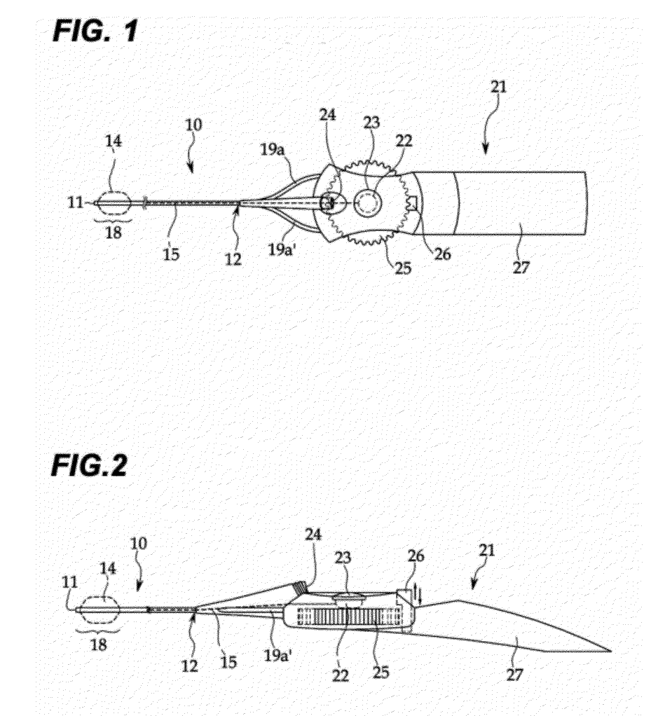

FIGS. 1 to 5 show a medical apparatus for dissociation of epidural adhesion and alleviation of stenosis of the intervertebral foramen according to a first exemplary embodiment of the present disclosure.

As shown in FIGS. 1 and 2, the medical apparatus of the present disclosure includes an insertion tube 10 and a main body 21.

Insertion tube 10 includes a first end 11 to be inserted in vivo and a second end 12 that is opposite to first end 11. Main body 21 is connected to insertion tube 10 at second end 12 of insertion tube 10.

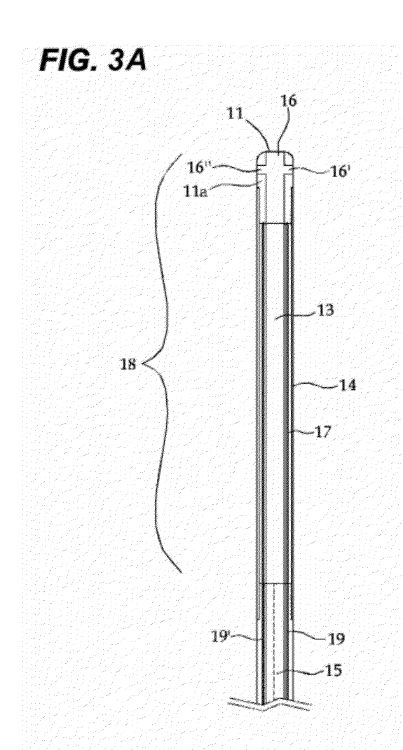

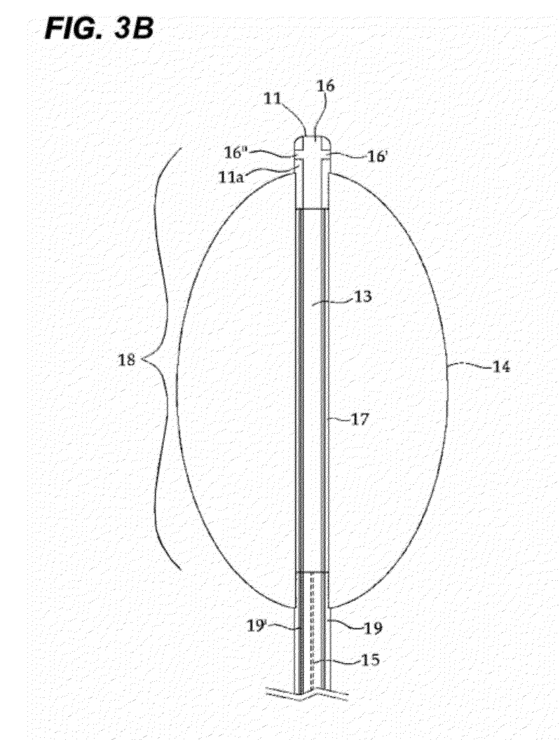

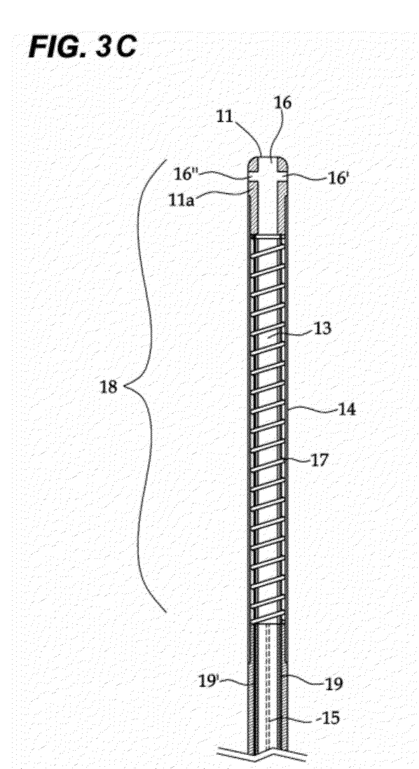

As shown magnified in FIGS. 3A and 3A, a through hole 13 extending from first end 11 to second end 12 is formed inside insertion tube 10.

As shown in FIGS. 3A and 3B, insertion tube 10 may include an inflatable balloon part 14 around first end 11 to be inserted in vivo. More specifically, balloon part 14 is formed spaced apart at a predetermined distance from first end 11 of insertion tube 10 on an outer side of insertion tube 10.

In particular, as shown in FIG. 3A, balloon part 14 may be formed to have a side identical to the outer side of insertion tube 10. When balloon part 14 is formed to have a side identical to the outer side of insertion tube 10, in vivo tissues are not damaged or in vivo entrapment of insertion tube 10 does not occur during insertion of insertion tube 10.

Balloon part 14 is inflatable as a fluid (for example, air) is supplied from a fluid supply source. FIG. 3A shows a state where the fluid is not supplied, while FIG. 3B shows a state where the fluid is supplied to inflate balloon part 14. In order to supply the fluid to balloon part 14, a fluid injection tube 15 is installed inside a wall of insertion tube 10 along a longitudinal direction of insertion tube 10. Fluid injection tube 15 allows balloon part 14 to communicate with the fluid supply source to supply the fluid to balloon part 14.

The fluid supply source communicating with fluid injection tube 15 may employ a syringe including the fluid. However, as an example of the fluid supply source, a flexible fluid bag 22 including a fluid may be contemplated. Fluid bag 22 and balloon part 14 may be connected to each other through fluid injection tube 15 to supply the fluid to balloon part 14 by an amount decreased from the volume of fluid bag 22 and thus balloon part 14 may be formed to be inflated. In this way, a desired expansion of balloon part 14 may be obtained by controlling the pressurized volume of fluid bag 22.

Fluid bag 22 may be formed in main body 21, and a button 23 may be formed on main body 21 such that fluid bag 22 may be pressed. Thus, the apparatus has an advantage in that it is possible to manipulate the apparatus with one hand because main body 21 may be gripped and button 23 may be pressed to pressurize fluid bag 22 and control the expansion of balloon part 14.

Balloon part 14 is effective in physically alleviating epidural adhesion and stenosis of the intervertebral foramen. This physical alleviation may be allowed by inserting the first end 11 of insertion tube 10 in vivo during surgery and then inflating balloon part 14.

In particular, if either one of insertion tube 10 and balloon part 14 or both are formed of a radio-opaque material or designed to include radio-opaque elements, for example, a thin steel wire, it is very effective because insertion tube 10 and balloon part 14 may be seen on a radiograph in a radiographic equipment during surgery.

Although air is typically used as a fluid contained in fluid bag 22, a contrast agent may be used as a fluid that inflates balloon part 14. The term “contrast agent” refers to a chemical used to enhance the contrast of images by enlarging the difference in X-ray absorption of each tissue artificially such that tissues or vessels may be well observed during a radioactive examination. A contrast agent may be used to confirm the degree of expansion of balloon part 14 with radiographic equipment when balloon part 14 is inflated by injecting the contrast agent.

An embodiment in which an external fluid supply unit 200, which will be described below in a second exemplary embodiment, is mounted in main body 21 instead of fluid bag 22 may be contemplated (an illustration therefore will be omitted here). That is, an injector connected to fluidly communicate with fluid injection tube 15 may be mounted in main body 21, a slidably movable piston in the injector may extend to the outside of main body 21, and a spring may be inserted between the injector and the piston such that the spring may push the piston in a direction of increasing an internal volume of the injector.

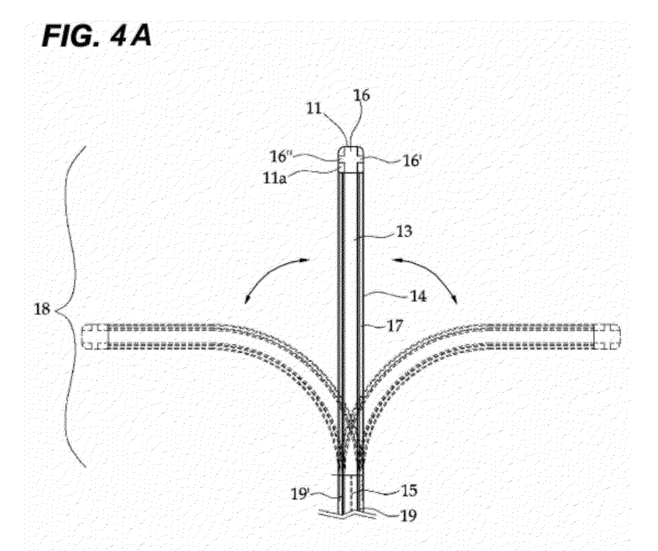

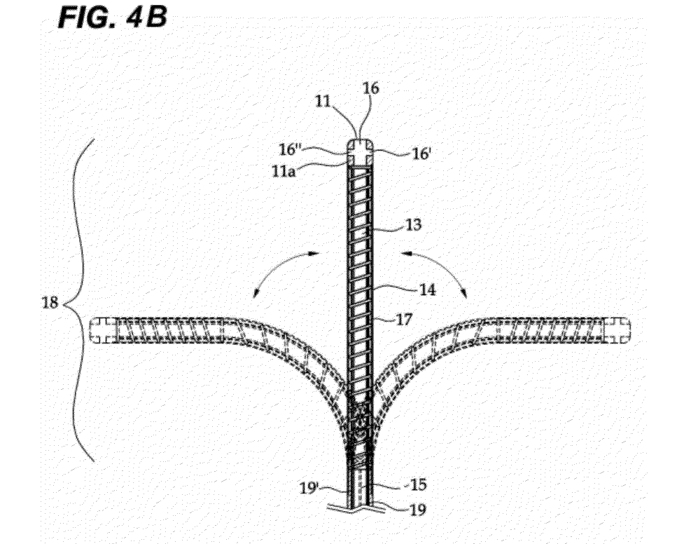

As shown in FIG. 4A, a movable end 18 may be formed around first end 11 of insertion tube 10 so as to dissociate epidural adhesion and alleviate stenosis of the intervertebral foramen.

In order to manipulate movable end 18, a pair of wires 19 and 19′ are installed. More specifically, the pair of wires 19 and 19′ are attached around first end 11 of insertion tube 10. The pair of wires 19 and 19′ extend toward second end 12 of insertion tube 10 and may extend through a wall of insertion tube 10 such that the wires may not be exposed to the outside.

When one of the pair of wires, for example, a right wire 19 is pulled, only a right portion of insertion tube 10 contracts to bend movable end 18 to the right side. When the other of the pair of wires, for example, a left wire 19′ is pulled, a left portion of insertion tube 10 also contracts to bend movable end 18 to the left side.

In particular, the balloon part is formed of a flexible material, compared to the insertion tube, and thus a portion of insertion tube 10 from first end 11 of the insertion tube to the balloon part serves as movable end 18. For dissociation of epidural adhesion and alleviation of stenosis of the intervertebral foramen, balloon part 14 and movable end 18 are disposed in a site of insertion tube 10, which may be physically manipulated, that is, in a position where balloon part 14 and movable end 18 overlap with each other, and thus the inflation of balloon part 14 or the manipulation of movable end 18 may be selectively performed if necessary.

As shown in FIG. 3C, a spring 17 may be additionally installed inside balloon part 14. In particular, when a coil spring represented as spring 17 is used as shown in the figure, a through hole 13 of insertion tube 10 may not only pass the inside of the coil spring, but may also be maintained without being decreased by the coil spring even though the movable end 18 is bent. Thus, the coil spring can be used for inserting a catheter C or administering a drug.

When spring 17 is installed, movable end 18 operates as shown in FIG. 4B. Since spring 17 is installed, movable end 18 easily returns to an original position by a restoring force of spring 17 if none of the wires are pulled.

As shown in FIGS. 1 and 2, the pair of wires 19 and 19′ extend, for example, through connection tubes 19a and 19a′ to main body 21 to be attached to a dial 25 which is rotatably installed in main body 21. Thus, any one of the pair of wires 19 and 19′ may be selectively pulled according to a rotation of dial 25 in one direction. Accordingly, movable end 18 of insertion tube 10 may be easily manipulated by gripping a handle portion 27 of main body 21 and rotating dial 25. Irregularities may be formed on the perimeter of dial 25 so as to prevent manipulation errors such as slipping.

A fixing switch 26 is installed in main body 21 such that the switch may fix the rotation of dial 25. Fixing switch 26 may be engaged or disengaged with irregularities of the dial according to the up and down movement of fixing switch 26. When movable end 18 of insertion tube 10 is manipulated by fixing switch 26, if necessary, the rotation of dial 25 may be fixed in an appropriate position to maintain the bent state of movable end 18.

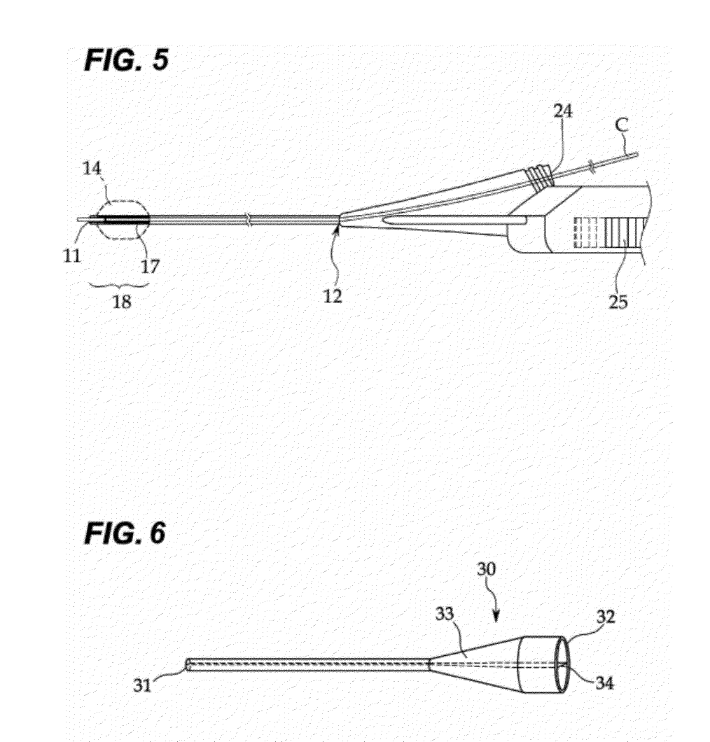

As shown in FIG. 5, through hole 13 of insertion tube 10 may function as a passage for directly injecting a drug, and as a guide passage for inserting a catheter C for drug injection. Holes 16, 16′, and 16″ for spraying a drug, communicating with through hole 13, are formed at first end 11 of insertion tube 10 and a side wall 11a around the first end. Holes 16, 16′, and 16″ for spraying a drug are effective in spreading the drug uniformly in many directions through holes 16, 16′, and 16″ for spraying a drug.

A conduit 24 provided in the main body is connected to second end 12 of insertion tube 10, and thus the drug may be injected or the catheter C may be inserted through conduit 24 from main body 21 into through hole 13 of insertion tube 10. Conduit 24 may be positioned at a front portion of main body 21, that is, adjacent to a portion where insertion tube 10 and main body 21 are connected to each other. Conduit 24 may be positioned at a front portion of main body 21 to decrease the length of insertion tube 10 to be inserted, and may be positioned at a front side of handle portion 27 of main body 21 to facilitate a surgery work for the injection of the drug or the insertion of the catheter C through conduit 24.

When the medical apparatus is used, after balloon part 14 or movable end 18 is manipulated to perform physical dissociation, insertion tube 10 may be pulled at a predetermined distance, that is, by a distance between balloon 14 or movable end 18 and first end 11 of insertion tube 10, and then the drug may be injected directly or by using the catheter C to effectively administer the drug to a site of the physical dissociation. Thus, the medical apparatus is useful.

The medical apparatus according to the first exemplary embodiment of the present disclosure described above is intended to dissociate epidural adhesion and alleviate stenosis of the intervertebral foramen by inserting an insertion tube 10 into the epidural space through the caudal vertebrae, but it is not limited thereto.

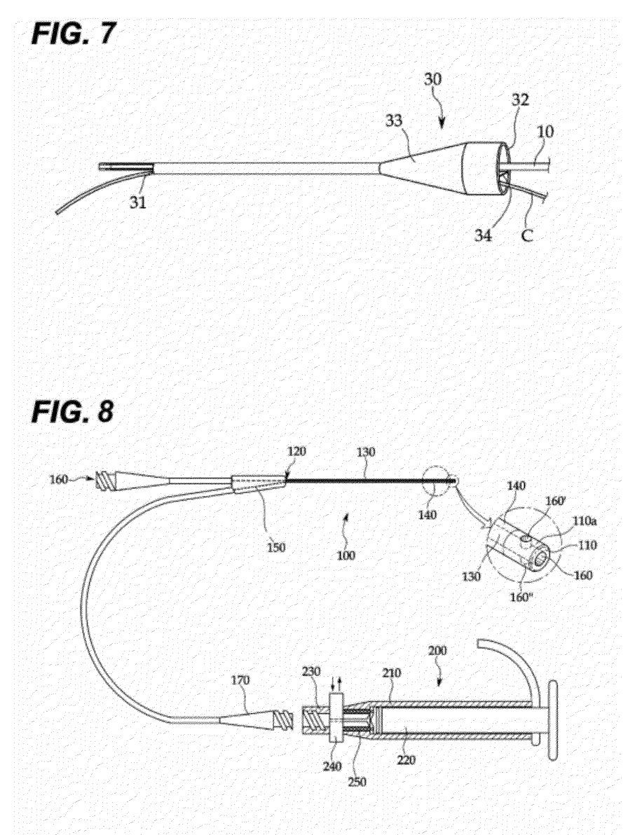

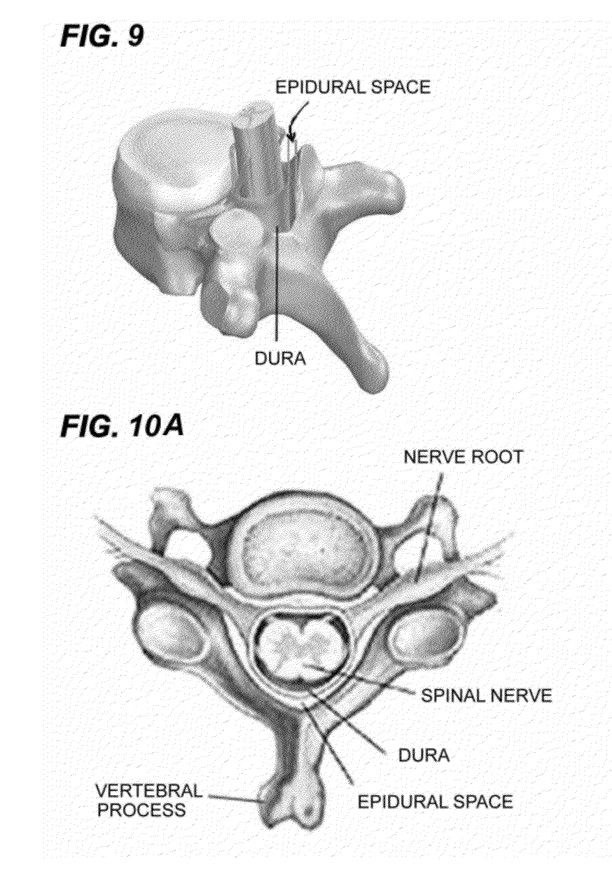

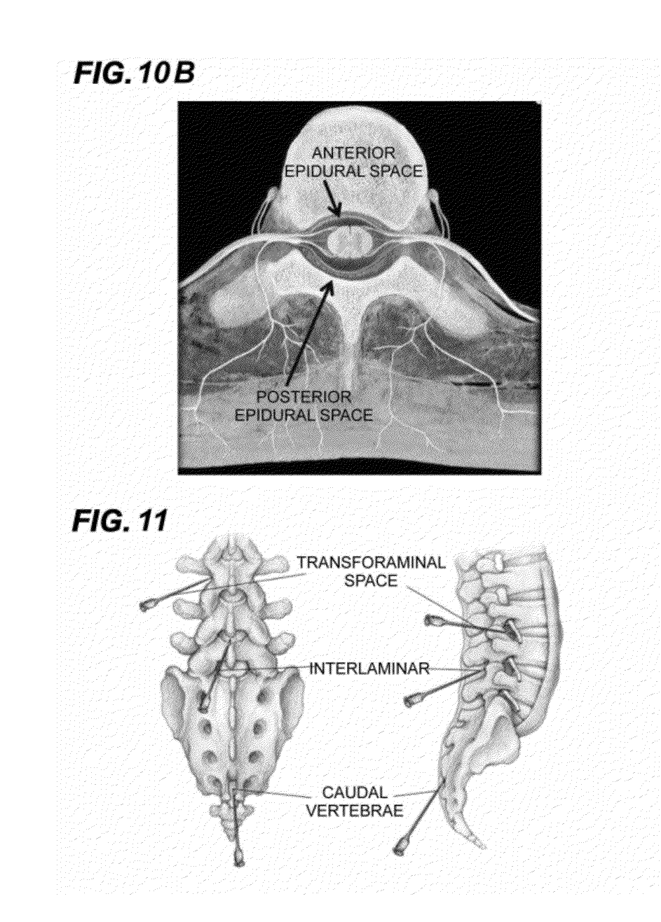

Next, FIGS. 6 and 7 show an insertion tube guide 30 for guiding insertion tube 10 such that two or more catheters C may be inserted. It is effective to administer a drug to an affected area actually during surgery while two or more catheters C are inserted into a human body and left therein for 3 to 5 days. Thus, insertion tube guide 30 may be used such that two or more catheters C may be inserted in vivo and left therein.

Insertion tube guide 30 includes a first end 31 to be inserted in vivo, a second end 32 that is opposite to first end 31, an external wall 33 progressively narrowing from second end 32 to first end 31, and one or more diaphragms 34 dividing an internal space extending from second end 32 to first end 31.

When first end 31 is inserted in vivo, a long internal space is divided into two or more ones by diaphragm(s) 34. Thus, one insertion tube 10 may be first inserted into one of the internal spaces to leave a catheter C therein, and then another insertion tube 10 may be inserted into the other space to leave another catheter C therein. Here, diaphragm 34 is formed of a flexible material and thus may be modified when insertion tube 10 is inserted. When insertion tube guide 30 is used, a plurality of catheters do not interfere with each other and thus the insertion tube guide 30 is useful in inserting the plurality of insertion tubes 10.

FIG. 8 shows a medical apparatus according to a second exemplary embodiment of the present disclosure. The difference of the second exemplary embodiment from the first exemplary embodiment is that a catheter 100 is directly used instead of an insertion tube for guiding the catheter and an end of the catheter is not bent in the second exemplary embodiment.

More specifically, according to the second exemplary embodiment, a medical apparatus includes catheter 100, and catheter 100 includes a first end 110 to be inserted in vivo and a second end 120 that is opposite to first end 110, and a through hole 130 extending from first end 110 to second end 120 is formed therein.

A balloon part 140, which is inflatable as a fluid is supplied, is formed at an outer side of catheter 100 spaced apart at a predetermined distance from first end 110 of catheter 100. As in the previous embodiment, balloon part 140 may be formed to have a side identical to the outer side of catheter 100 so as to prevent in vivo tissues from being damaged or in vivo entrapment of catheter 100 from occurring when catheter 100 is inserted.

A fluid injection tube 150 may be installed inside the wall of catheter 100 along a longitudinal direction of catheter 100, and fluid injection tube 150 allows balloon part 140 to communicate with a fluid supply source to supply the fluid to balloon part 140 and thus inflate balloon part 140.

Holes 160, 160′, and 160″ for spraying a drug, communicating with through hole 130, are formed at first end 110 of catheter 100 and a side wall 110a around first end 110, and a drug injection port 180 may be connected to second end 120 of catheter 100 such that drug injection port 180 may communicate with through hole 130 of catheter 100. Thus a drug is directly injected into a desired site.

Of course, although a syringe storing a fluid may be used as a fluid supply source, external fluid supply unit 200 may be contemplated as a fluid supply source to communicate with fluid injection tube 150 in the second exemplary embodiment.

First, fluid injection tube 150 extends from second end 120 of catheter 100 to the outside of the catheter, and a connecting part 170 to be connected to external fluid supply unit 200 is formed at an extended end of fluid injection tube 150.

External fluid supply unit 200 includes an injector 210, a piston 220 slidably movable within the injector 210, and a connecting part 230 that is detachable to connecting part 170 of the fluid injection tube. Through this configuration, piston 220 of the external fluid supply unit 200 may be controlled to preset the internal volume of external fluid supply unit 200 before connecting part 230 of external fluid supply unit 200 is connected to fluid injection tube 150. Of course, external fluid supply unit 200 and balloon part 140 are configured to be connected through fluid injection tube 150 to each other and a fluid is supplied to balloon part 140 by an amount decreased from the internal volume of external fluid supply unit 200 to inflate balloon part 140.

In external fluid supply unit 200, a spring 250 may be inserted, for example, between piston 220 and injector 210. Spring 250 pushes piston 220 in a direction of increasing the internal volume of external fluid supply unit 200. Thus, balloon part 140 is maintained not to be inflated at normal times, and only when the piston is pressed to decrease the internal volume of external fluid supply unit 200, a fluid is supplied to balloon part 140 to start the inflation of balloon part 140. Air or a contrast agent may be used as a fluid for inflating balloon part 140.

External fluid supply unit 200 may further include a blocking valve 240 capable of blocking communication with fluid injection tube 150, and the blocking valve may move in an arrow direction as shown to perform fluid communication and blocking. Fluid injection tube 150 and external fluid supply unit 200 are advantageous in that the volume of the fluid in external fluid supply unit 200 may be accurately preset by putting the fluid into external fluid supply unit 200 and blocking the fluid using blocking valve 240 before fluid injection tube 150 and external fluid supply unit 200 are connected to each other, and that an inflated state of balloon part 140 may be maintained by blocking fluid communication at a state where balloon part 140 is inflated after fluid injection tube 150 and external fluid supply unit 200 are connected to each other.

As in the first exemplary embodiment, either one of catheter 100 and balloon part 140 or both may be considered to be formed of a radio-opaque material or include radio-opaque elements.

The medical apparatus according to the second exemplary embodiment of the present disclosure described above is mainly intended to dissociate epidural adhesion and alleviate stenosis of the intervertebral foramen by inserting catheter 100 through the transforaminal space, but it is not limited thereto.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A medical apparatus for dissociation of epidural adhesion and alleviation of stenosis of the intervertebral foramen, the apparatus comprising:
an insertion tube configured to include a first end to be inserted in vivo and a second end that is opposite to the first end, wherein a through hole extending from the first end to the second end is formed therein;
a balloon part formed on an outer side of the insertion tube, being spaced apart at a predetermined distance from the first end of the insertion tube and configured to be inflatable as a fluid is supplied; and
a fluid injection tube installed in a wall of the insertion tube along a longitudinal direction of the insertion tube and configured to supply the fluid to the balloon part by communicating the balloon part with a fluid supply source.

2. The medical apparatus of claim 1, further comprising:
a pair of wires configured to be attached around the first end of the insertion tube and configured to be extended toward the second end of the insertion tube,
wherein a portion of the insertion tube from the first end of the insertion tube to the balloon part forms a movable end, and
the movable end is bent to one side when one of the pair of wires is pulled, and the movable end is bent to the other side when the other of the pair of wires is pulled.

3. The medical apparatus of claim 2, wherein the pair of wires extend through an inside of a wall of the insertion tube.

4. The medical apparatus of claim 2, further comprising:
a main body configured to be connected to the insertion tube at the second end of the insertion tube; and
a dial which is rotatably connected to the main body and to which a pair of wires are connected,
wherein the pair of wires are attached to the dial such that any one of the pair of wires is selectively pulled according to rotation in one direction of the dial.

5. The medical apparatus of claim 2, further comprising a fixing switch installed on the main body and configured to fix a rotation of the dial.

6. The medical apparatus of claim 1, wherein the through hole of the insertion tube is for injecting a drug or inserting a catheter.

7. The medical apparatus of claim 1, wherein holes for spraying a drug, communicating with the through hole, are formed at the first end of the insertion tube and a side wall around the first end of the insertion tube.

8. The medical apparatus of claim 1, further comprising:
a main body configured to be connected to the insertion tube at the second end of the insertion tube,
wherein a conduit provided in the main body is connected to the second end of the insertion tube and the conduit is used for injecting a drug or inserting a catheter into a through hole of the insertion tube.

9. A medical apparatus for dissociation of epidural adhesion and alleviation of stenosis of the intervertebral foramen, the apparatus comprising:
a catheter configured to include a first end to be inserted in vivo and a second end that is opposite to the first end, wherein a through hole extending from the first end to the second end is formed therein;
a balloon part formed on an outer side of the catheter, being spaced apart at a predetermined distance from the first end of the catheter and configured to be inflatable as a fluid is supplied; and
a fluid injection tube installed in a wall of the catheter along a longitudinal direction of the catheter and configured to supply the fluid to the balloon part by communicating the balloon part with a fluid supply source.

10. The medical apparatus of claim 9, wherein holes for spraying a drug, communicating with the through hole, are formed at the first end of the catheter and a side wall around the first end of the catheter.

11. The medical apparatus of claim 9, wherein a drug injection port is connected to the second end of the catheter such that the drug injection port communicates with the through hole of the catheter.

12. The medical apparatus of claim 9, wherein the fluid injection tube extends from the second end of the catheter to the outside of the catheter and a connecting part to be connected to the fluid supply source is formed at an extended end of the fluid injection tube.
